(19) United States
(12) Patent Application Publication
(10) Pub. No.: US 2002/0082457 A1
(43) Pub. Date: Jun. 27, 2002

(54) Process for the preparation of 1-(2,2,6-trimethylcyclohexyl)-3-alkanols

(76) Inventors: Walter Kuhn, Holzminden (DE); Horst Surburg, Holzminden (DE)

Correspondence Address: BAYER CORPORATION, PATENT DEPARTMENT, 100 BAYER ROAD, PITTSBURGH, PA, 15205, US

(21) Appl. No.: 10/015,320

(22) Filed: Dec. 12, 2001

(30) Foreign Application Priority Data
Dec. 15, 2000 (DE) 10062771.4

(51) Int. Cl. C07C 29/14

(52) U.S. Cl. 568/881

(57) ABSTRACT

The present invention relates to a process for the preparation of 1-(2,2,6-trimethylcyclohexyl)-3-alkanols with a content of at least 15% of trans isomers, based on the total amount of the 1-(2,2,6-trimethylcyclohexyl)-3-alkanol, by catalytic hydrogenation, where a) 1-(2,6,6-trimethyl-1 or 2-cyclohexen-1-yl)-1-alken-3-ones are reacted in the presence of ruthenium-containing catalysts or catalyst mixtures, or b) 1 -(2,6,6-trimethyl-1 or 2-cyclohexen-1-yl)-1-alken-3-ols are reacted in the presence of catalysts which contain 1 or more elements of groups Ib, VIb and VIII of the Periodic Table of the Elements.

### FIELD OF THE INVENTION

The present invention relates to a process for the preparation of 1-(2,2,6-trimethylcyclohexyl)-3-alkanols with an increased content of trans isomers.

### BACKGROUND OF THE INVENTION

1-(2,2,6-Trimethylcyclohexyl)-3-alkanols of the general formula

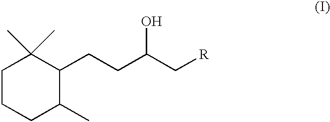

are valuable odorants which, because of their characteristic woody odor and their good fixing action, are widely used in the preparation of perfume compositions (DE 2 455 761 A1 and DE 2 807 584 A1). These compounds are readily accessible via the hydrogenation of the corresponding 1-(2,6,6-trimethyl-1 or 2-cyclohexen-1-yl)-1-alken-3-ones of the formula

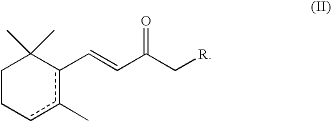

These unsaturated ketones can be prepared in a relatively simple technical manner by the condensation of citral with the corresponding 2-alkanones and subsequent cyclization (Bauer, Garbe, Surburg, Common Fragrance and Flavor Materials, 3rd Ed., Wiley-VCH, Weinheim 1997, p61 ff.).

However, the hydrogenated products are isomer mixtures of the formula III and IV in which the actual odor-imparting component is the trans isomer (cf. EP 118 809 A2):

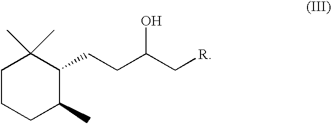

trans-1-(2,2,6-Trimethylcyclohexyl)-3-alkanols, R is methyl or ethyl.

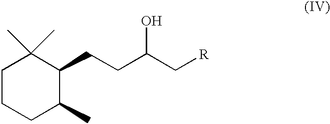

cis-1-(2,2 ,6-Trimethylcyclohexyl )-3-alkanols, R is methyl or ethyl.

However, in the process procedures given in DE 2 455 761 A1 and DE 2 807 584 A1, Helv. Chim. Acta 26 (1943), p. 2151 ff. [catalytic hydrogenation using Raney nickel, copper chromite or platinum catalysts], the trans isomer is formed only to a minor extent; no more than about 10-12% of the total amount of the 1-(2,2,6-trimethylcyclohexyl)-3-alkanols present have the valuable trans configuration.

For this reason, various processes have been developed which describe the targeted preparation of the trans-1-(2,2,6-trimethylcyclohexyl)-3-alkanols (e.g. EP 118 809 A2, 118 817 A1 and 456 932 B1).

However, because of their multistage nature or the use of difficult-to-handle reagents such as lithium aluminum hydride, the processes described are unsuitable for implementation on a relatively large industrial scale.

### SUMMARY OF THE INVENTION

Therefore, it was an object of the present invention to provide an industrial process which is easy to carry out and which produces 1-(2,2,6-trimethylcyclohexyl)-3-alkanols with an increased proportion, i.e. at least 15%, of trans isomers.

We have found a process for the preparation of 1-(2,2,6-trimethylcyclohexyl)-3-alkanols with a content of at least 15% of trans isomers, based on the total amount of the 1-(2,2,6-trimethylcyclohexyl)-3-alkanol, by catalytic hydrogenation, which is characterized in that

a) 1-(2,6,6-trimethyl-1 or 2-cyclohexen-1-yl)-1-alken-3-ones are reacted in the presence of ruthenium-containing catalysts or catalyst mixtures, or

b) 1-(2,6,6-trimethyl-1 or 2-cyclohexen-1-yI)-1-alken-3-ols are reacted in the presence of catalysts which contain one or more elements of groups Ib, VIb and VIII of the Periodic Table of the Elements.

Surprisingly, in one embodiment of the present invention, it has been found that the catalytic hydrogenation of the 1-(2,6,6-trimethyl-1 or 2-cyclohexen-1-yl)-1-alken-3-ones using ruthenium catalysts leads to trans/cis mixtures of 1-(2,2,6-trimethylcyclohexyl)-3-alkanols with a content of trans isomers which is higher than that which can be achieved using the processes described in the prior art. The processes described in the prior art lead to a trans content of at most about 10%, based on the total amount of 1-(2,2,6-trimethylcyclohexyl)-3-alkanol present, corresponding to a trans/cis ratio of not more than 1:7.5. The hydrogenation using ruthenium catalysts leads, by contrast, to a trans content of more than 15%, based on the total amount of 1-(2,2,6-trimethylcyclohexyl)-3-alkanol present, corresponding to a trans/cis ratio of more than 1:5.5. The trans content here is preferably about 20%.

The ruthenium used as catalyst is preferably applied to a support. Preference is given here to materials such as activated carbon, aluminum oxide or silicon dioxide. The concentration of the ruthenium catalyst on the support material is in the range from 5 to 10% by weight. The ruthenium can also be generated before the start of or during the hydrogenation in situ by reduction with hydrogen from ruthenium tetroxide or ruthenium tetroxide hydrate, and optionally be precipitated onto one of the above described supports. The weight ratio of ruthenium catalyst used to the 1-(2,6,6-trimethyl-1 or 2-cyclohexen-1-yl)-1-alken-3-ones is preferably 0.001 to 0.1:1, more preferably 0.01 to 0.05:1.

The hydrogen pressure during the hydrogenation can be 1 to 100 bar, more preference is given to a range from 10 to 20 bar. The temperature during the hydrogenation can be between 20 and 250° C. Preference is given to a range between 100 and 160° C. More preference is given to a range between 130 and 150° C.

The hydrogenation can be carried out either without a diluent or else in solution. Suitable solvents are substances such as alcohols, e.g. ethanol, such as ethers, e.g. tetrahydrofuran and ethylene glycol dimethyl ether, such as esters, e.g. ethyl acetate, and such as hydrocarbons, e.g. hexane and cyclohexane.

As well as having the advantage of producing products with the desired higher trans proportion, the use of ruthenium catalysts according to the present invention also has the positive effect of leading to complete conversion. In contrast to the processes described in the prior art, only small amounts of unreacted starting material are found in the hydrogenation product. This makes final purification of the hydrogenation product to an odor-sensorily perfect quality considerably easier.

Furthermore, in a second embodiment of the invention, it has surprisingly been found that the catalytic hydrogenation of the 1-(2,6,6-trimethyl-1 or 2-cyclohexen-1-yl)-1-alken-3-ols of the formula V

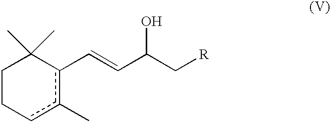

(R=methyl, ethyl) leads to a mixture of the 1-(2,2,6-trimethylcyclohexyl)-3-alkanols with an increased content of trans isomers. In this process variant, it is possible to achieve an even higher content of trans isomers than in the first variant. The trans/cis ratio can here preferably be a trans content of more than 30%, based on the total amount of 1-(2,2,6-trimethylcyclohexyl)-3-alkanol present, corresponding to a trans/cis ratio of more than 1:2. The trans content is preferably between 30 and 40%.

The 1-(2,6,6-trimethyl-1 or 2-cyclohexen-1-yl)-1-alken-3-ol can be prepared in a manner known per se by reduction from 1-(2,6,6-trimethyl-1 or 2-cyclohexen-1-yl)-1-alken-1-one, respectively. The reduction here can be carried out using reducing agents known per se. These include, for example, sodium borohydride.

The catalysts used for the hydrogenation of the 1-(2,6,6-trimethyl-1 or 2-cyclohexen-1-yl)-1-alken-3-ols are, for example, those described, for example, in Houben-Weyl, Methoden der Organischen Chemie [Methods of organic chemistry], Vol. IV/1c, pp.18-28. Preference is given to using hydrogenation catalysts which contain one or more elements of groups Ib, VIb and VII of the Periodic Table of the Elements, in particular copper, chromium, cobalt, rhodium, ruthenium, nickel, palladium and platinum.

The catalysts used according to the present invention are generally applied to a support. Preference is given in this respect to materials such as activated carbon, aluminum oxide or silicon dioxide. The concentration of the catalysts on the support material is preferably in the range from 5 to 10%.

The catalysts used according to the present invention can also be generated prior to the start of or during the hydrogenation in situ by reduction with hydrogen from the corresponding oxides or salts, and optionally precipitated onto one of the above described supports.

The weight ratio of catalyst used to the 1-(2,6,6-trimethyl-1 or 2-cyclohexen-1-yl)-1-alken-3-ols is preferably 0.001 to 0.1:1. More preference is given to a ratio of from 0.01 to 0.05:1.

The hydrogen pressure during the hydrogenation can be 1 to 100 bar, preference is given to a range between 1 and 20 bar, and more preference given to a range between 5 and 15 bar. The temperature during the hydrogenation can be between 20 and 250° C., preference is given to a range between 20 and 100° C., and more preference is given to a range between 20 and 50° C.

The hydrogenation can either be carried out without a diluent or in solution. Suitable solvents are substances such as alcohols, e.g. ethanol, such as ethers, e.g. tetrahydrofuran and ethylene glycol dimethyl ether, such as esters, e.g. ethyl acetate, and such as hydrocarbons, e.g. hexane and cyclohexane.

The examples below illustrate the process according to the present invention in more detail. However, the use of the process according to the present invention is not limited to these examples.

### EXAMPLE 1

2540 g of a mixture of methyl ionones, which comprises the n-methylionones 1-(2,6,6-trimethyl-2-cyclohexen-1-yl)-1-penten-3-one (so-called α-n-methylionone) and 1-(2,6,6-trimethyl-1-cyclohexen-1-yl)-1-penten-3-one (so-called β-n-methylionone) in a ratio of about 4:1, are introduced with 52 g of ruthenium catalyst 5% on activated carbon (50% water content) into a stirred autoclave with gas-dispersion stirrer. The hydrogen absorption takes place over the course of 60 h at 136-140° C. and 20 bar. Following filtration and distillation, 2320 g of product are obtained which comprise the 1-(2,2,6-trimethylcyclohexyl)-3-pentanols in a trans/cis ratio of 1:4.5.

### EXAMPLE 2

258 g of a mixture of 1-(2,6,6-trimethyl-1-cyclohexen-1-yl)-1-penten-3-ol and 1-(2,6,6-trimethyl-2-cyclohexen-1-yl)-1-penten-3-ol, which has been obtained from the n-methylionones-containing mixture described in Example 1 by reduction with sodium borohydride, 750 ml of ethanol and 5 g of palladium on activated carbon (5%) are introduced into a 2 l stirred autoclave. The hydrogen absorption takes place at about 10 bar between 25 and 50° C. over the course of 6 h. After filtration and distillation, 220 g of product are obtained which comprise the 1-(2,2,6-trimethylcyclohexyl)-3-pentanols in a trans/cis ratio of 1:2.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for the preparation of 1-(2,2,6-trimethylcyclohexyl)-3-alkanols with a content of at least 15% of trans isomers, based on the total amount of the 1-(2,2,6-trimethylcyclohexyl)-3-alkanol, by catalytic hydrogenation, comprising the step of reacting 1-(2,6,6-trimethyl-1 or 2-cyclohexen-1-yl)-1-alken-3-ones in the presence of ruthenium-containing catalysts or catalyst mixtures.

2. A process according to claim 1, wherein the catalysts are prepared before or during the hydrogenation by reduction with hydrogen in situ from the oxides or salts of the catalyst metals.

3. A process according to claim 1, wherein the weight ratio of the catalysts to 1-(2,6,6-trimethyl-1 or 2-cyclohexen-1-yl)-1-alken-3-ones is between 0.001:1 and 0.1:1.

4. A process according to claim 3, wherein the weight ratio of the catalysts to 1-(2,6,6-trimethyl-1 or 2-cyclohexen-1-yl)-1-alken-3-ones is between 0.001:1 and 0.5:1.

5. A process according to claim 1, wherein the catalysts are applied to supports.

6. A process according to claim 5, wherein the catalysts are applied to activated carbon, aluminum oxide or silicon dioxide as support material.

7. A process according to one of claim 5, wherein the concentration of the catalyst on the support material is between 5 and 10%, based on the support material.

8. A process according to claim 2, wherein reduction is carried out at a pressure between 1 and 100 bar.

9. A process according to claim 1, wherein hydrogenation is carried out at temperatures between 20 and 250° C.

10. A process for the preparation of 1-(2,2,6-trimethylcyclohexyl)-3-alkanols with a content of at least 15% of trans isomers, based on the total amount of the 1-(2,2,6-trimethylcyclohexyl)-3-alkanol, by catalytic hydrogenation, comprising the step of reacting 1-(2,6,6-trimethyl-1 or 2-cyclohexen-1-yl)-1-alken-3-ols in the presence of catalysts which contain one or more elements of groups Ib, VIb and VIII of the Periodic Table of the Elements.

11. A process according to claim 10, wherein the catalysts are prepared before or during hydrogenation by reduction with hydrogen in situ from the oxides or salts of the catalyst metals.

12. A process according to claim 10, wherein said catalysts are selected from the group consisting of copper, chromium, cobalt, rhodium, ruthenium, nickel, palladium, platinum and mixtures of these metals.

13. A process according to claim 10, wherein the weight ratio of the catalysts to 1-(2,6,6-trimethyl-1 or 2-cyclohexen-1-yl)-1-alken-3-ols is between 0.001:1 and 0.1:1.

14. A process according to claim 13, wherein the weight ratio of the catalysts to 1-(2,6,6-trimethyl-1 or 2-cyclohexen-1-yl)-1-alken-3-ols is between 0.01:1 and 0.05:1.

15. A process according to claim 10, wherein the catalysts are applied to supports.

16. A process according to claim 15, wherein the catalysts are applied to activated carbon, aluminum oxide or silicon dioxide as support material.

17. A process according to claim 15, wherein the concentration of the catalyst on the support material is between 5 and 10%, based on the support material.

18. A process according to claim 10, wherein 1-(2,6,6-trimethyl-1 or 2-cyclohexen-1-yl)-1-alken-3-ol is first prepared by reducing 1-(2,6,6-trimethyl-1 or 2-cyclohexen-1-yl)-1-alken-3-one.

19. A process according to claim 18, wherein reduction is carried out by adding sodium borohydride.

20. A process according to claim 10, wherein reduction is carried out at a pressure between 1 and 100 bar.

21. A process according to claim 10, wherein hydrogenation is carried out at temperatures between 20 and 250° C.

22. A mixture comprising cis and trans isomers of 1-(2,2,6-trimethylcyclohexyl)-3-alkanols, wherein the content of trans isomers is more than 15%.

23. A mixture according to claim 22, wherein the trans isomer content is ≧20%.

24. A perfumed product comprising a mixture of cis and trans isomers of 1-(2,2,6-trimethylcyclohexyl)-3-alkanols, wherein the content of trans isomers is more than 15%.
